(19) United States
(12) Patent Application Publication
(10) Pub. No.: US 2011/0257396 A1
(43) Pub. Date: Oct. 20, 2011

(54) PROCESS FOR THE MANUFACTURE OF CIS(-)-LAMIVUDINE

(75) Inventors: Girij Pal Singh, Maharashtra (IN); Dhananjai Srivastava, Maharashtra (IN); Srinivas Ayyalasomayajula Satya, Maharashtra (IN); Manmeet Brijkishore Saini, Maharashtra (IN); Harishchandra Sambhaji Jadhav, Maharashtra (IN); Aparna Murlidharan Warrier, Maharashtra (IN); Nilesh Bhimsingh Dumre, Maharashtra (IN)

(73) Assignee: Lupin Limited, Mumbai (IN)

(21) Appl. No.: 12/513,015

(22) PCT Filed: Sep. 10, 2007
(86) PCT No.: PCT/IN2007/000399

§ 371 Date: Jun. 28, 2011

(30) Foreign Application Priority Data
Oct. 30, 2006 (IN) 1144/KOL/2006

(51) Int. Cl. C07D 411/04

(52) U.S. Cl. 544/317

(57) ABSTRACT

An improved process for the manufacture of Lamivudine. The process involves:

(a) resolution of racemic lamivudine (intermediate of formula IX)

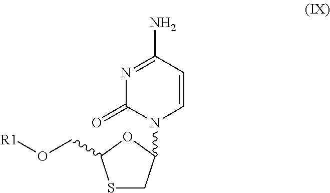

to cis (±) lamivudine of formula (XII) by forming a crystalline salt and separating the product from an organic solvent by fractional crystallization;

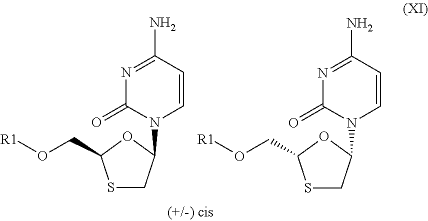

(b) resolution of cis (±) lamivudine to cis (−) isomer involving formation of S-Binol adduct of formula XIV.

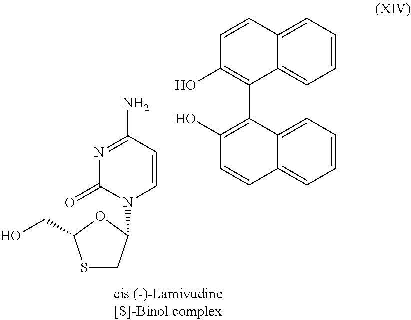

### FIELD OF INVENTION

The present invention relates to an improved process for the Manufacture of Lamivudine.

### BACKGROUND OF THE INVENTION

Lamivudine (I) (CAS No. 134678-17-4) is chemically known as (−)-[2R,5S]-4-amino-1-[2-(hydroxymethyl)-1,3-oxathiolan-5-yl]-2(1H)-pyrimidin-2-one.

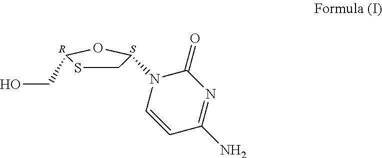

Lamivudine is a reverse transcriptase inhibitor used alone or in combination with other classes of Anti-HIV drugs in the treatment of HIV infection. It is available commercially as a pharmaceutical composition under the brand name EPIVIR®, marketed by GlaxoSmithKline, and is covered under U.S. Pat. No. 5,047,407.

This molecule has two stereo-centres, thus giving rise to four stereoisomers: (±)-Cis Lamivudine and (±)-Trans Lamivudine. The pharmaceutically active isomer however is the (−)-Cis isomer which has the absolute configuration [2R,5S] as show in Formula (I).

U.S. Pat. No. 5,047,407 discloses the 1,3-oxathiolane derivatives; their geometric (cis/trans) and optical isomers. This patent describes the preparation of Lamivudine as a mixture of cis and trans isomers (shown in scheme I). The diastereomers obtained are converted into N-acetyl derivatives before separation by column chromatography using ethylacetate and methanol (99:1); however, this patent remains silent about further resolution of the cis isomer to the desired (−)-[2R,5S]-Cis-Lamivudine. Secondly, as the ethoxy group is a poor leaving group, the condensation of cytosine with compound VI gives a poor yield, i.e. 30-40%, of compound VII. Thirdly, chromatographic separation that has been achieved only after acetylation requires a further step of de-acetylation of the cis-(±)-isomer. Also, separation of large volumes of a compound by column chromatography makes the process undesirable on a commercial scale.

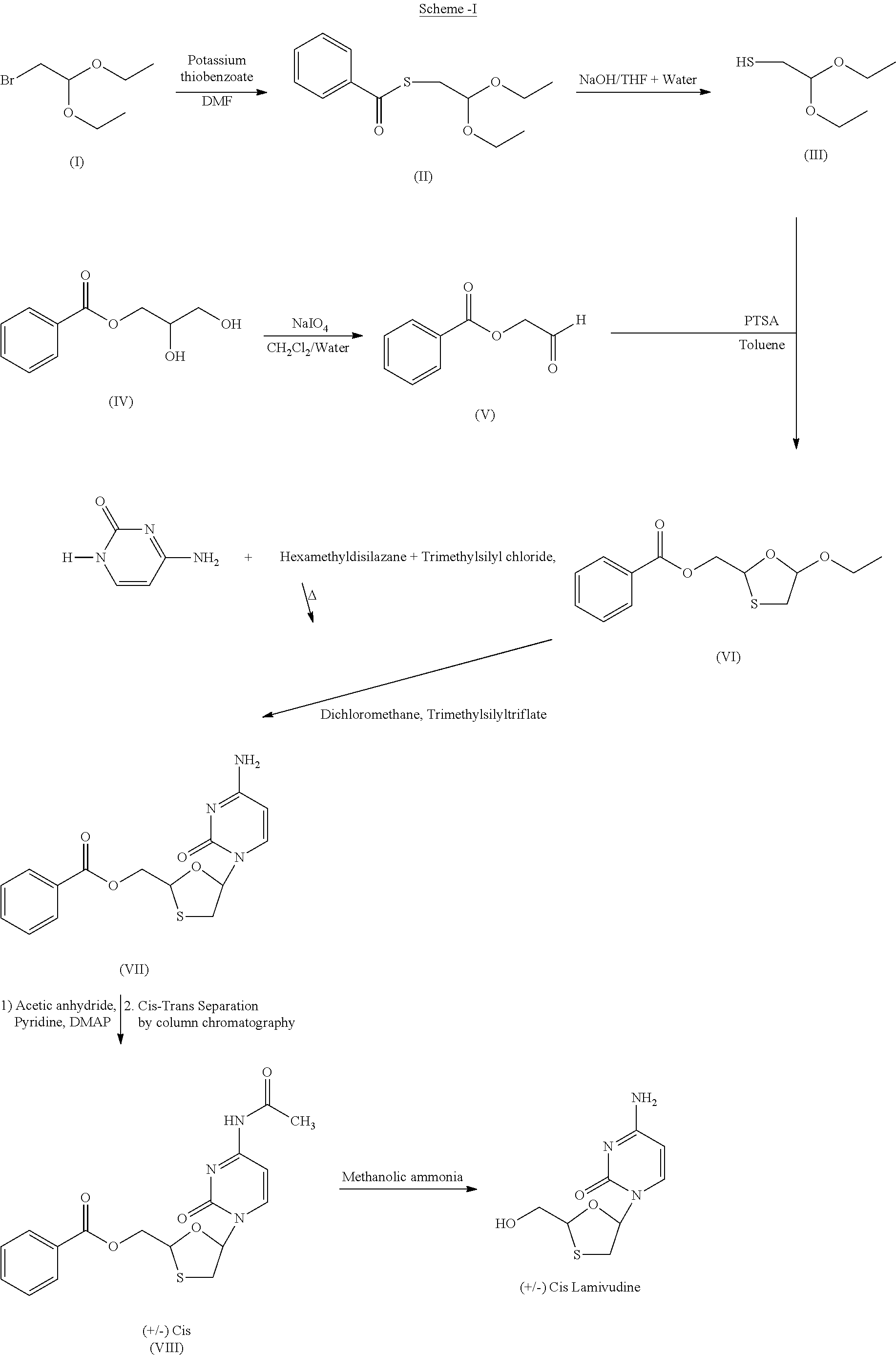

Efforts have been made in the past to overcome the shortcomings of low yield and enantiomeric enrichment. In general, there have been two approaches to synthesize (−)-[2R,5S]-Cis-Lamivudine. One approach involves stereoselective synthesis, some examples of which are discussed below.

U.S. Pat. No. 5,248,776 describes an asymmetric process for the synthesis of enantiomerically pure β-L-(−)-1,3-oxathiolone-nucleosides starting from optically pure 1,6-thioanhydro-L-gulose, which in turn can be easily prepared from L-Gulose. The condensation of the 1,3-oxathiolane derivative with the heterocyclic base is carried out in the presence of a Lewis acid, most preferably SnCl₄, to give the [2R,5R] and [2R,5S] diastereomers that are then separated chromatographically.

U.S. Pat. No. 5,756,706 relates a process where compound A is esterified and reduced to compound B. The hydroxy group is then converted to a leaving group (like acetyl) and the cis- and trans-2R-tetrahydrofuran derivatives are treated with a pyrimidine base, like N-acetylcytosine, in the presence trimethylsilyl triflate to give compound C in the diastereomeric ratio 4:1 of cis and trans isomers.

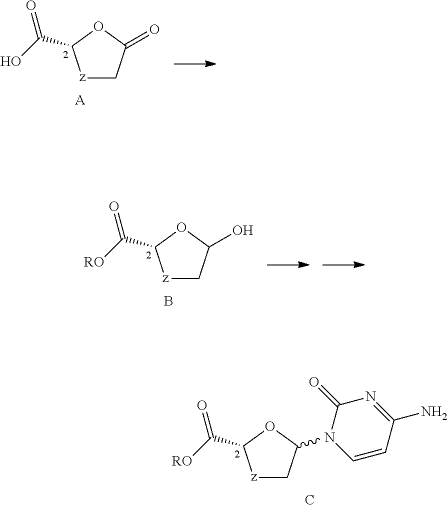

Z=S, CH

Dissolving compound C in a mixture of 3:7 ethyl acetate-hexane separates the cis isomer. The product containing predominantly the cis-2R,5S isomer and some trans-2R,5R compound is reduced with NaBH₄ and subjected to column chromatography (30% MeOH-EtOAc) to yield the below compound.

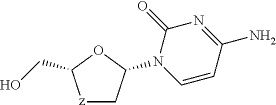

U.S. Pat. No. 6,175,008 describes the preparation of Lamivudine by reacting mercaptoacetaldehyde dimer with glyoxalate and further with silylated pyrimidine base to give mainly the cis-isomer by using an appropriate Lewis acid, like TMS-I, TMS-Tf, TiCl₄ et cetera. However the stereoselectivity is not absolute and although the cis isomer is obtained in excess, this process still requires its separation from the trans isomer. The separation of the diastereomers is done by acetylation and chromatographic separation followed by deacetylation. Further separation of the enantiomers of the cis-isomer is not mentioned.

U.S. Pat. No. 6,939,965 discloses the glycosylation of 5-fluoro-cytosine with compound F (configuration: 2R and 2S)

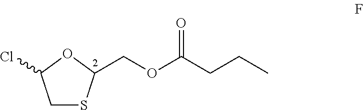

The glycosylation is carried out in the presence of TiCl₃(OiPr) which is stereoselective and the cis-2R,5S-isomer is obtained in excess over the trans-2S,5S-isomer. These diastereomers are then separated by fractional crystallization.

U.S. Pat. No. 6,600,044 relates a method for converting the undesired trans-1,3-oxathiolane nucleoside to the desired cis isomer by a method of anomerization or transglycosylation and the separation of the hydroxy-protected form of cis-, trans-(−)-nucleosides by fractional crystallization of their hydrochloride, hydrobromide, methanesulfonate salts. However, these cis-trans isomers already bear the [R] configuration at C2 and only differ in their configuration at C5; i.e. the isomers are [2R,5R] and [2R,5S]. Hence diastereomeric separation directly yields the desired [2R,5S] enantiomer of Lamivudine.

In the second approach to prepare enantiomerically pure Lamivudine the resolution of racemic mixtures of nucleosides is carried out. U.S. Pat. No. 5,728,575 provides one such method by using enzyme-mediated enantioselective hydrolysis of esters of the formula

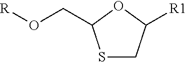

wherein, ‘R’ is an acyl group and ‘R1’ represents the purine or pyrimidine base. ‘R’ may be alkyl carboxylic, substituted alkyl carboxylic and preferably an acyl group that is significantly electron-withdrawing, eg. α-haloesters. After selective hydrolysis, the process involves further separation of the unhydrolyzed ester from the enantiomerically pure 1,3-oxathiolane-nucleoside. Three methods are suggested in this patent, which are:
1. Separation of the more lipophilic unhydrolyzed ester by solvent extraction with one of a wide variety of nonpolar organic solvents.
2. Lyophilization followed by extraction into MeOH or EtOH.
3. Using an HPLC column designed for chiral separations.

In another of its aspects, this patent also refers to the use of the enzyme cytidine-deoxycytidine deaminase, which is enantiomer-specific, to catalyze the deamination of the cytosine moiety and thereby converting it to uridine. Thus, the enantiomer that remains unreacted is still basic and can be extracted by using an acidic solution.

However, the above methods suffer from the following drawbacks. (a) Enzymatic hydrolysis sets down limitations on choice of solvents: alcohol solvents cannot be used as they denature enzymes.

(b) Lyophilization on an industrial scale is tedious. (c) Chiral column chromatographic separations are expensive.

WO 2006/096954 describes the separation of protected or unprotected enantiomers of the cis nucleosides of below formula by using a chiral acid to form diastereomeric salts that are isolated by filtration. Some of the acids used are R-(−)-Camphorsulfonic acid, L-(−)-Tartaric acid, L-(−)-Malic acid, et cetera. However, the configuration of these CIS-nucleosides are [2R,4R] and [2S,4S] as the heterocyclic base is attached at the 4 position of the oxathiolane ring and the overall stereo-structure of the molecule changes from that of the 2,5-substituted oxathiolane ring.

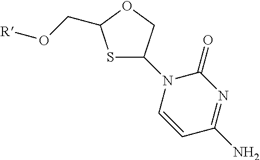

Thus various methods are described for the preparation of Lamivudine. However there is no mention in the prior art about the separation of an enantiomeric pair, either cis-(±) or trans-(±), from a mixture containing cis-[2R,5S], [2S,5R] and trans-[2R,5R], [2S,5S] isomers. Further, there also is a need to provide resolution of the cis-(±) isomers to yield the desired enantiomer in high optical purity.

CN 1223262 (Deng et al) teaches the resolution of a certain class of compounds called Prazoles by using chiral host compounds such as dinaphthalenephenols (BINOL), diphenanthrenols or tartaric acid derivatives. The method consists of the formation of a 1:1 complex between the chiral host (BINOL) and one of the enantiomers, the guest molecule. The other enantiomer remains in solution. (S)-Omeprazole, which is pharmaceutically active as a highly potent inhibitor of gastric acid secretion, has been isolated from its racemic mixture in this manner by using S-BINOL.

BINOL is a versatile chiral ligand that has found its uses in various reactions involving asymmetric synthesis (Noyori, R. Asymmetric Catalysis in Organic Synthesis) and optical resolution (Cram, D. J. et al J. Org. Chem. 1977, 42, 4173-4184). Some of these reactions include BINOL-mediated oxidation and reduction reactions, C—C bond formation reactions such as Aldol reaction, Michael addition, Mannich reaction et cetera (Brunel Chem. Rev. 2005 105, 857-897) and kinetic resolution, resolution by inclusion complexation et cetera.

BINOL, or 1,1′-bi-2-Naphthol, being an atropoisomer possesses the property of chiral recognition towards appropriate compounds. One of the uses of BINOL in resolution that is known in literature is in Host-Guest complexation. In one such example, 1,1-binaphthyl derivatives have been successfully incorporated into optically active crown ethers for the enantioselective complexation of amino acid esters and chiral primary ammonium ions (Cram, D. J. Acc. Chem. Res. 1978, 11, 8-14). The chiral ‘host’ is thus able to discriminate between enantiomeric compounds by the formation of hydrogen bonds between the ether oxygen and the enantiomers. The complex formed with one of the isomers, the ‘guest’, will be less stable on steric grounds and this forms the basis for its separation.

It is evident from the literature cited that there exists a need to (a) synthesize Lamivudine by a process requiring less expensive, less hazardous and easily available reagents, and (b) achieve good yields with superior quality of product without resorting to column chromatography as a means of separation, thereby making the process of Lamivudine manufacture more acceptable industrially.

### OBJECT OF THE INVENTION

Thus, one object of the present invention is to provide a process for the synthesis of Lamivudine which is cost effective, uses less hazardous and easily available reagents, yet achieves good yields with superior quality of product without resorting to column chromatography.

A further object of the present invention is to provide an improved process for the synthesis of Lamivudine, by separating the mixture of diastereomers: Cis-[2R,5S], [2S,5R] from Trans-[2R,5R], [2S,5S] and then resolving the Cis isomers using BINOL to obtain (−)-[2R,5S]-Cis-Lamivudine with at least 99% ee.

### SUMMARY OF THE INVENTION

Thus, according to one aspect of the present invention there is provided a process to separate the Cis-Trans diastereomeric mixture of the intermediate IX based on the difference in solubility of the diastereomers or their salts with an acid in a suitable solvent, the process comprising the following steps:
a. providing the cis-trans mixture of hydroxy-protected Lamivudine of Formula (IX) as starting material, the different isomers having the configurations Cis-[2R,5S], [2S,5R] and Trans-[2R,5R], [2S,5S],
b. treating the hydroxy-protected intermediate IX, in an organic solvent with an acid to form its corresponding salt,
c. filtering the above solution to isolate the cis diastereomer, i.e. salt of hydroxy-protected-Cis-(±)-Lamivudine,
d. converting the above salt to the free base, i.e. hydroxy-protected-Cis-(±)-Lamivudine,
e. carrying out a deprotection step to get Cis(±)-Lamivudine.

According to another aspect of the invention, there is provided a process for the preparation of an optically pure or optically enriched enantiomer of Lamivudine of Formula (I), the process comprises of—
a. providing a mixture of optical isomers i.e. Cis(±)-Lamivudine, the different enantiomers having the configuration [2R,5S] and [2S,5R],
b. reacting the mixture of optical isomers with a chiral host in an organic solvent,
c. separating the adduct formed by the enantiomer and the chiral host,
d. treating the adduct with an acid and then neutralizing it to get (−)-[2R,5S]-Cis-Lamivudine,
e. optionally purifying it by crystallization from a suitable organic solvent, thereby obtaining the (−)-[2R,5S]-Cis-Lamivudine in a substantially optically pure or optically enriched form.

Thus, the problems associated with chromatographic separations have also been eliminated as the separation of isomers, both diastereomeric and enantiomeric, is done by selective crystallization.

### DETAILED DESCRIPTION OF THE INVENTION

The process of the present invention for the manufacture of Lamivudine is as presented in Scheme 2, and comprises reacting compound IV

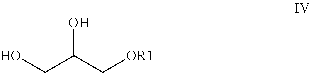

where, R1 is tert-butyldiphenylsilyl or benzoyl
with sodium periodate to yield compound V. Compound V is then condensed with 2-mercaptoacetaldehyde dimer and subsequently acetylated with acetyl chloride to give the protected 2-hydroxymethyl-1,3-oxathiolan-5-yl acetate (compound VIII). This 1,3-oxathiolane compound VIII is further condensed with silylated cytosine in the presence of a Lewis acid such as trimethylsilyliodide to get protected 6-amino-3-{2-hydroxymethyl-1,3-oxathiolan-5-yl}-3-hydropyrimidine-2-one (compound IX).

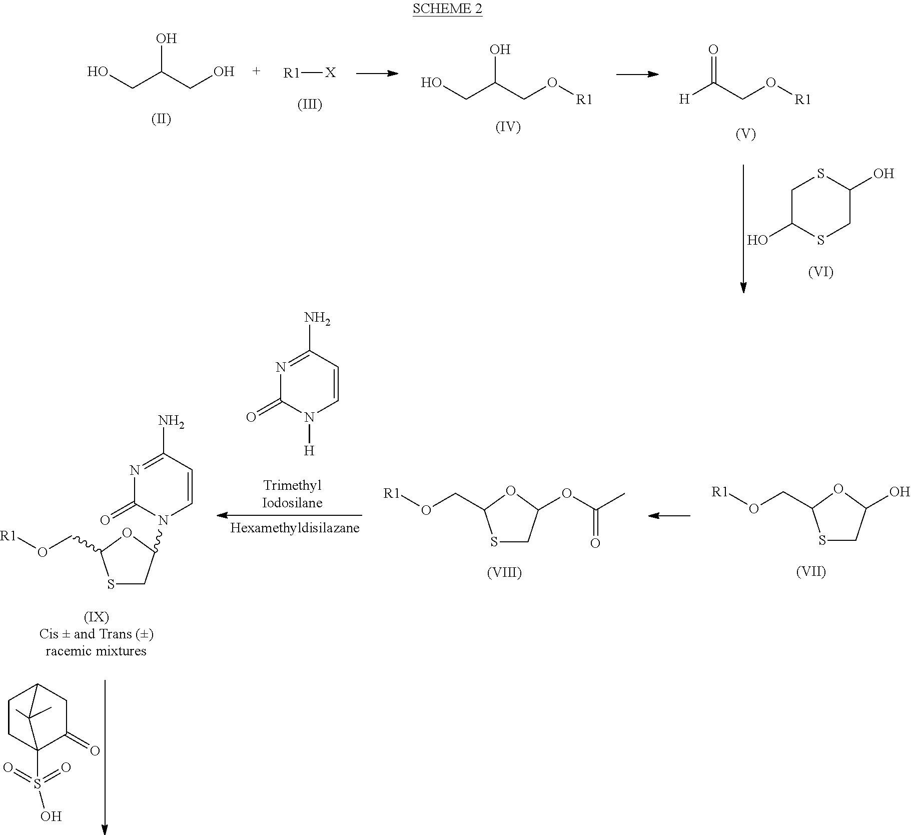

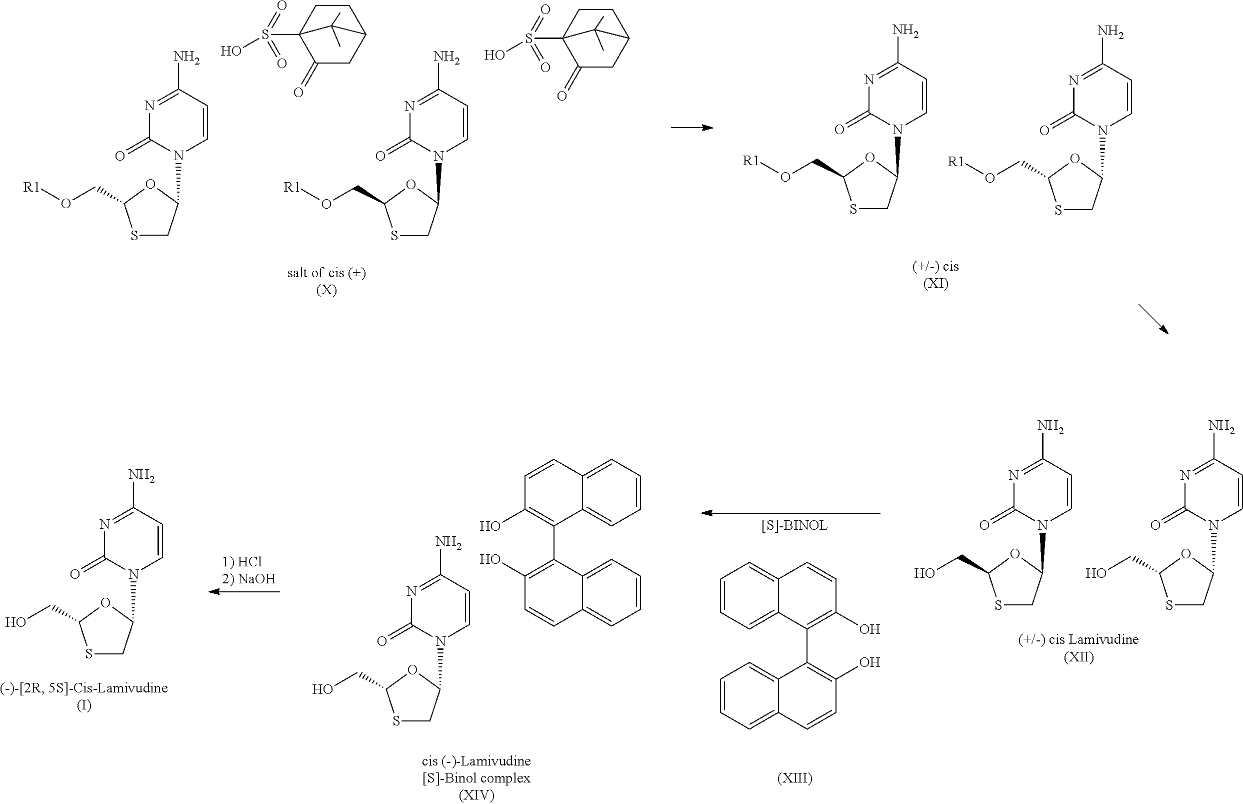

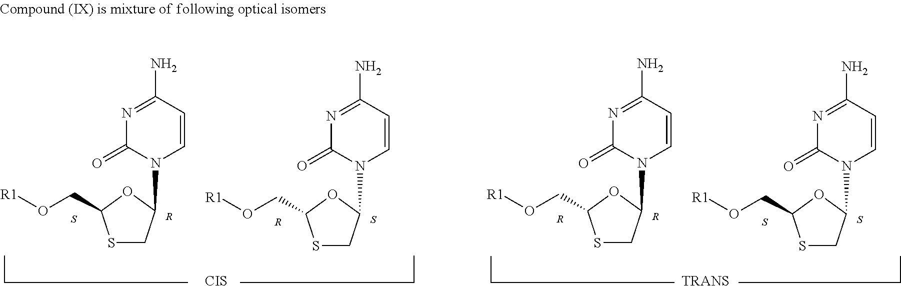

The separation of the four-component diastereomeric mixture of isomers bearing the following configuration: trans-[2R,5R], [2S,5S] and cis-[2R,5S], [2S,5R] forms the next step. The separation efficiency of the benzoyl-protected compound IX diastereomers i.e. cis-(±) from trans-(±) from their solution in methanol or dichloromethane was found to be poor. In the former case, the compound is highly soluble in methanol whereas in the latter, despite getting the cis racemates with a purity of 97.7%, the yield was found to be only 36.44% of material balance.

In one embodiment of the present invention, the benzoyl-protected compound IX is treated with various achiral and chiral acids like succinic acid, oxalic acid, [S]-(+)-mandelic acid and di-para-toluoyl-D-Tartaric acid in a hydroxylic solvent like methanol to give the corresponding four diastereomer salts. Interestingly, the inventors have found that the Cis-(±)-isomer salts alone precipitate from the solution with a diastereomeric purity in almost all cases [excepting di-para-toluoyl-D-Tartaric acid which has 87%] greater than 98.5% at first pass with an accompanying yield of 40.8%, 28.86%, 50.52% and 27.04% respectively of overall material balance. The Cis-(±)-isomers are obtained exclusively because of the formation of a eutectic, leaving behind in solution the trans-(±)-isomers.

In another embodiment of the present invention the tert-butyldiphenylsilyl-protected compound IX when treated with 1S-(+)-camphorsulfonic acid in a hydroxylic solvent like methanol also permits the separation of the Cis-(±)-isomers. The solid product isolated is composed of only the [2S,5R] and [2R,5S] i.e. the Cis-(±)-isomers with 88.36% diastereomeric purity and 89% material balance at first pass. After re-crystallizing the product twice with about 45 v/wt % MeOH, the diastereomeric purity increases to 98.9%.

The same separation using 1S-(+)-camphorsulfonic acid has also been attempted with other solvents and solvent systems like ethyl acetate and dichloromethane. It can be seen that the separation efficiency is a function of the solvent and the acid used as the following table indicates.

TABLE 1
Separation of TBDSi-protected Cis-(±)-isomers
using 1S-(+)-CSA in different solvents

| SOLVENT | V/WT % | CIS % |
|---|---|---|
| Ethyl acetate | 5 | 74.5 |
| Ethyl acetate/DCM | 5 | 84.6 |
| DCM | 5 | 88.04 |
| MeOH | 4 | 88.36 |

The separation of the cis racemates from the trans racemates was also tried using other acids such as Di-para-toluoyl-D-Tartaric acid in IPA and in ethyl acetate, D-(−)-Tartaric acid in IPA, L-(+)-Tartaric acid in IPA, Naproxen in ethyl acetate and N-(carbobenzyloxy)-L-Phenyl alanine in ethyl acetate. No solid product enabling the diastereomeric separation was obtained with any of these salts.

Cis-(±)-isomers that are isolated are first converted to their free base and further deprotected to get racemic cis-lamivudine (compound XII) having the configurations [2R,5S] and [2S,5R]. Thus, at this stage in the process, the complete separation of two components from the four-component mixture of Cis-[2R,5S], [2S,5R] and Trans-[2R,5R], [2S,5S] has been successfully accomplished.

Another aspect of the present invention provides a method for the separation of (−)-[2R,5S]-Cis-Lamivudine from its (+)-enantiomer using optically pure (S)-2,2′-dihydroxy-1,1′-binaphthyl [(S)-BINOL].

The process is operationally simple and comprises the following steps:

a) treating the racemates with (S)-BINOL in the presence of a hydroxylic solvent like methanol,
b) isolating the adduct formed by the enantiomer and the chiral host by filtration,
c) if desired, crystallizing the adduct,
d) separating the guest from the host by using an acid, preferably concentrated HCl, neutralizing the solution to get the free base and purifying it by recrystallization;
thereby yielding the desired (−)-[2R,5S]-Lamivudine with an enantiomeric excess greater than 99%.

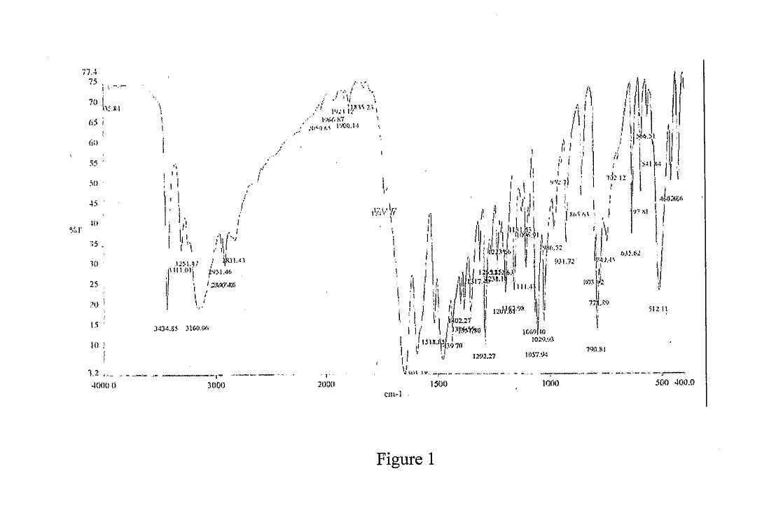

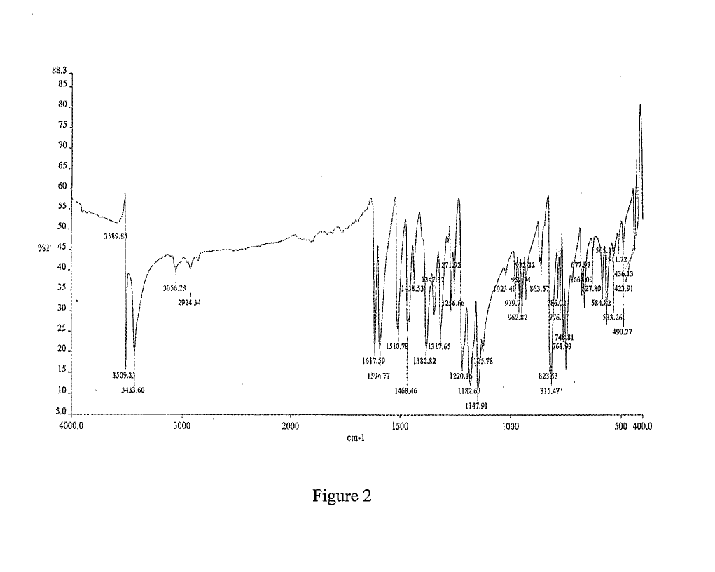

Thus, reacting the racemates with (S)-BINOL results in the formation of a clathrate of (−)-[2R,5S]-Cis-Lamivudine by Host-Guest complexation. On filtration it affords a clean separation between the enantiomers. In the final step, the (−)-[2R,5S]-Cis-Lamivudine-(S)-BINOL complex is broken in an acidic medium and then neutralized to obtain the desired product: (−)-[2R,5S]-Lamivudine, in high optical purity of greater than 97.5% before purification. Re-crystallization with isopropanol raises the enantiomeric excess to 99.5%. The IR spectra of racemic Lamivudine, S-BINOL and the Lamivudine—S-BINOL complex are provided in FIGS. 1, 2, and 3 respectively. It is to be emphasized that when the same separation was attempted using R-BINOL, no resolution of enantiomers was achieved.

Resolution of the Cis enantiomers of Lamivudine by the formation of diastereomeric salts of cis-(±) lamivudine with acids like malic acid, mandelic acid, dibenzoyl tartaric acid, 3-bromocamphor-8-sulfonic acid, 10-camphorsulfonic acid, and di-p-toluoyltartaric acid have been attempted before by Liotta et al, one of the inventors named in U.S. Pat. No. 5,204,466. However, these attempts were unsuccessful as revealed by a declaration attached with the prosecution history file of U.S. Pat. No. 6,703,396.

Interestingly, the inventors have found that when the salts Cis-(±)-Lamivudine were prepared with R-(−)—CSA or with D-(−)-tartaric acid, no separation of enantiomers was achieved.

The following examples illustrate the practice of the invention without being limiting in any way. Example 1

### Preparation of 3-(2,2-dimethyl-1,1-diphenyl-1-silapropoxy)propane-1,2-diol (Tbdpsi-Glycerol)

### Compound IV

300 g (1.09 moles) of tert-butyldiphenylsilylchloride was added slowly over a period of 60 to 120 minutes at 0-5° C. to a solution of 110 g (1.19 moles) of Glycerol and 329.3 g (3.26 moles, 453 mL) of triethylamine in 300 mL of dimethylformamide. After stirring for 6 hours it was added to 1.5 L of cooled (5° C.-10° C.) DM water. 500 mL of DCM was added to this solution with stirring at room temperature and the layers were separated. To the aqueous layer 500 mL of DCM was added and the layers were separated again. The organic layers were combined and washed with a 25% aqueous solution of NaCl. The organic layer was isolated and the solvent was recovered under vacuum. 400 mL of hexane was added to the reaction mixture which was then cooled to 0-5° C. for 60 to 70 minutes. The solution was filtered to isolate the solid which was then washed with 100 mL of hexane and dried under vacuum at 40-45° C. Yield: 200-220 g (55.7-61.3%)

m.p.=91.7-92.9° C.

MS: M⁺-1=329

¹H NMR (CDCl₃): δ 1.07 (s, 9H), 3.64-3.71 (m, 4H), 3.73-3.81 (m, 1H), 7.35-7.68 (m, 10H)

### Example 2

### Preparation of 2-(2,2-dimethyl-1,1-diphenyl-1-silapropoxy)ethanal (TBDPSi-aldehyde)

### Compound V

To TBDPSi-glycerol (200 g, 0.605 moles) in a solution of 2.7 L acetone and 300 mL DM water, 168 g (0.786 moles) of NaIO₄ was added in portions. The reaction mixture was stirred for 2 hours and then filtered at the end of the reaction. The solid was washed with 400 mL of DCM and the solvent was recovered under vacuum at 40-45° C. 1600 mL DCM was added to the concentrate followed by 1 L of DM water. The organic layer was separated and the solvent recovered under vacuum at 45-50° C. Yield: 170-175 g (94.4-97.2%)

¹H NMR (CDCl₃): δ 1.12 (s, 9H), 4.23 (s, 2H), 7.36-7.70 (m, 10H), 9.73 (s, 1H)

### Example 3

### Preparation of [2-(2,2-dimethyl-1,1-diphenyl-1-silapropoxy)methyl]-1,3-oxathiolan-5-yl acetate (TBDPSi-acetoxy oxathiolane)

### Compound VIII

170 g (0.57 moles) of TBDPSi-aldehyde was heated to 60-65° C. with 52 g (0.34 moles) of 1,4-dithiane-2,5-diol for 15-20 minutes in the presence of 188 ml of pyridine and stirred for 3 hours at that temperature. After completion of the reaction, the reaction mixture was cooled to 0-5° C. and then 850 mL of DCM was added to it. After stirring for 10-15 minutes, a mixture of 134.3 g (1.71 moles, 121.6 ml) of acetyl chloride in 340 mL of DCM was added slowly over a period of 60-90 minutes and stirred for 1 hour at 0-5° C. When the reaction was complete 850 mL of DM water was added to it at 5-10° C. and the reaction mixture was stirred for 10-15 minutes at 20-25° C. The organic layer was separated and washed with 850 mL of 5% aqueous solution of NaHCO₃. The organic layer was separated and the washing was repeated with 850 mL of 25% aqueous NaCl. The organic layer was separated again and the solvent was recovered under vacuum at 40-45° C. 340 mL of hexane was then added and the solution was vacuum distilled (40-45° C.) to remove the traces of DCM. 850 mL of hexane and 8.5 g of activated charcoal were added to the concentrate. The mixture was stirred and filtered through a celite bed. The bed was washed with 150 mL of hexane and the solvent was recovered under vacuum at 40-45° C. Yield: 210-230 g (88.0-96.6%)

¹H NMR (CDCl₃): δ 1.10 (s, 9H), 2.11 (s, 3H), 3.08-3.16 (d, 1H), 3.26-3.34 (dd, 1H), 3.78-3.97 (m, 2H), 5.26 (t, 1H), 6.66 (d, 1H), 7.36-7.74 (m, 10H)

MS: M⁺=357

### Example 4

### Preparation of 6-amino-3-{2-[(2,2-dimethyl-1,1-diphenyl-1-silapropoxy)methyl]-1,3-oxathiolan-5-yl)}-3-hydropyrimidine-2-one (TBDPSi-Cytosine)

### Compound IX

A mixture of 72.8 g (0.65 moles) of Cytosine, 527.5 mL (2.5 moles, 403.5 g) of Hexamethyldisilazane and 27.16 g (0.25 moles, 31 mL) of TMS-Cl was heated to 125-130° C. and refluxed for 2 hours in a nitrogen atmosphere. The reaction mixture was then cooled and the solvent was completely recovered under vacuum at 100-105° C. The residue was cooled to room temperature, dissolved in 2.1 L of DCM and 210 g (0.5 moles) of TBDPSi-acetoxy oxathiolane was added to it. 140 g (100 mL, 0.7 moles) of TMS-I was added to the reaction mixture slowly over 15-30 minutes. The reaction mixture was stirred for 4 hours. (14 mL of TMS-I can be added to the reaction mixture if the reaction has not reached completion.) It was then cooled to 0-5° C. and diluted with 1 L of DM water. After stirring for 15 minutes the organic layer was separated and washed with 1 L of aqueous NaHCO₃ solution. The organic layer was separated and the solvent was recovered under vacuum at 40-45° C. Yield: 200-230 g (85.0-93.6%)

MS: M+1=468

¹H NMR (CDCl₃): δ 1.05 (s, 9H), 3.15-3.17 (dd, 1H), 3.46-3.55 (dd, 1H), 3.92-3.98 (dd, 1H), 4.09-4.11 (dd, 1H), 5.21-5.24 (t, 1H), 5.72-5.76 (d, 1H), 6.07-6.35 (m, 1H)

### Example 5

### Preparation of 6-amino-3-{2-[(2,2-dimethyl-1,1-diphenyl-1-silapropoxy)methyl]-1,3-oxathiolan-5-yl)}-3-hydropyrimidine-2-one. CSA salt (TBDPSi-Cytosine.CSA salt)

### Compound X

450 mL of methanol was added to 210 g (0.5 moles) of TBDPSi-Cytosine and the solvent was recovered at 40-45° C. to remove the traces of DCM. The residue was diluted with 840 mL of MeOH and 105 g of (1S)-(+)-10-camphorsulfonic acid was added to it at room temperature. The mixture was stirred at room temperature for 6 hours and then filtered. The residue was washed with 210 mL of MeOH and dried under vacuum at 40-45° C. for 6 hours. 45 v/w % MeOH was added to the dried mass and was refluxed at 65-70° C. The solution was concentrated at atmospheric pressure to 10 v/w % and then filtered at room temperature. The residue was dried under vacuum at 40-45° C. for 6 hours. Yield: 121-130 g (38.0-41.26% w/w)

HPLC Purity Cis-(±)=97.3%

m.p.−190.8° C.

IR (in KBr, cm⁻¹): 3284, 3075, 2934, 1678, 1741, 1589, 1548, 1471, 1429, 1391, 1270, 1251, 1065, 1036.

### Example 6

### Preparation of 6-amino-3-{2-[(2,2-dimethyl-1,1-diphenyl-1-silapropoxy)methyl]-1,3-oxathiolan-5-yl)}-3-hydropyrimidine-2-one (TBDPSi-Cytosine free base)

### Compound XI

120 g (0.17 moles) of TBDPSi-Cytosine.CSA salt was treated with 100 mL of ammonia solution in the presence of 1.2 L of DCM and 600 mL of DM water at 30-35° C. (pH 9-10). The solution was stirred for 10-15 minutes and the layers were separated. The organic layer was washed with 300 mL of DM water, the organic layer was isolated again and the solvent was recovered under vacuum at 40-45° C. 200 mL of ethylacetate was added to the residue and the solvent was recovered under vacuum at 40-45° C. 400 mL of ethylacetate was added to it at 10-15° C. and the solution was filtered. The product was dried under vacuum at 40-45° C. for 6 hours. Yield: 70-75 g (87.8-93.93%)

m.p.: 200.3-205.1° C.

¹H NMR (CDCl₃): δ 1.05 (s, 9H), 3.05-3.12 (dd, 1H), 3.44-3.53 (dd, 1H), 3.87-3.95 (dd, 1H), 4.06-4.14 (dd, 1H), 5.19-5.26 (t, 1H), 5.52-5.56 (d, 1H), 6.27-6.31 (t, 1H), 7.25-7.67 (m, 10H), 7.91-7.95 (d, 1H)

MS: M+1=468

### Example 7

### Preparation of (+/−)-1-(2R/S-Cis)-4-amino-1-[(2-hydroxymethyl)-1,3-oxathiolan-5-yl]-2(1H)-pyrimidin-2-one (Lamivudine Cis±)

### Compound XII

To 70 g of TBDPSi-Cytosine free base dissolved in 700 mL of THF, 82 mL of TBAF (1M soln in THF) is added dropwise over 35-40 minutes at room temperature. The mixture is stirred for an hour and then filtered. 175 mL of ethanol-water mixture (EtOH:H₂O is 9:1) is added to the product, stirred for 1 hour and filtered. The residue is washed with 25 mL of ethanol-water mixture and dried under vacuum at 40-45° C. for 4-5 hours. Yield: 25-28 g (72.8-81.63%)

The IR spectrum of Cis-(±)-Lamivudine is given in FIG. 1.

¹H NMR (DMSO d₆): δ 2.98-3.07 (dd, 1H), 3.35-3.4 (dd, 1H), 3.71-3.73 (m, 2H), 5.14-5.18 (t, 1H) 5.34 (bs, 1H), 5.71-5.75 (d, 1H), 6.16-6.21 (t, 1H) 7.19-7.26 (d, 2H), 7.80-7.83 (d, 1H)

MS: M+1=230

### Example 8

### Preparation of (−)-1-(2R—Cis)-4-amino-1-[(2-hydroxymethyl)-1,3-oxathiolan-5-yl]-2(1H)-pyrimidin-2-one Binol complex (Lamivudine-BINOL complex)

### Compound XIV

To 25 g (0.1 moles) of Lamivudine (Cis+/−) dissolved in 250 mL of MeOH at 60-65° C., 37.5 g (0.131 moles) of S-(−)-BINOL was added. Once a clear solution was obtained it was allowed to cool to room temperature and was stirred for 6 hours. The solid was filtered and dried under vacuum at 40-45° C. for 4-5 hours. Yield: 20-22 g (74.6-82% w/w) Enantiomeric excess=97.93% which increases to 99.50% after purification. Purification was carried out by crystallizing the product from IPA containing 0.002 moles of [S]-BINOL.

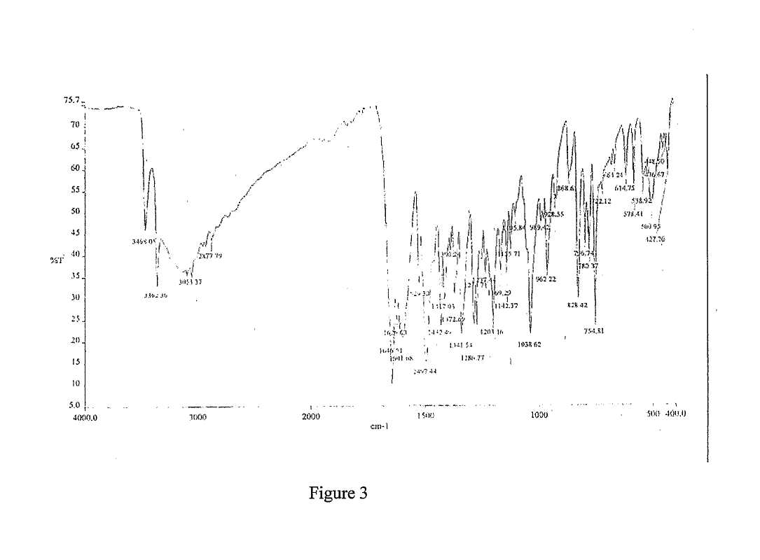

The IR spectrum of the complex is as given in FIG. 3. m.p.=190.1-190.6° C.

¹H NMR (DMSO d₆): 1.99-3.08 (dd, 1H), 3.36 (dd, 1H), 3.73 (m, 2H), 5.14-5.19 (t, 1H), 5.72-5.76 (d, 1H), 6.17-6.23 (t, 1H), 6.90-6.94 (d, 2H), 7.12-7.33 (m, 8H), 7.82-7.87 (m, 6H), 9.24 (bs, 2H)

XRD [2θ] (Cu-K_{α1}=1.54060 Å, K_{α2}=1.54443 Å K_{β}=1.39225 Å; 40 mA, 45 kV): 8.02, 10.52, 12.73, 14.33, 14.51, 16.06, 17.06, 17.80, 20.12, 21.94, 22.36, 23.70, 24.06 and 25.05.

### Example 9

### Preparation of Lamivudine: (−)-[2R,5S]-4-amino-1-[2-(hydroxymethyl)-1,3-oxathiolan-5-yl]-2(1H)-pyrimidin-2-one

### Compound I

5 mL of conc. HCl was slowly added to a solution of 20 g of Lamivudine-BINOL complex in 100 ml of ethylacetate and 100 mL of DM water (pH 2-2.5). The layers were separated and a 100 mL aliquot of ethylacetate was added to the aqueous layer. The layers were separated again and the aqueous layer was neutralized using 10 mL of 10% aqueous NaOH solution. The solvent was recovered under vacuum at 40-45° C., the product obtained was dissolved in 160 mL of methanol, filtered, the filtrate was concentrated and 32 mL of water-ethanol mixture (3:1) was added to this product, heated to get a clear solution, cooled to 5-10° C. and then filtered. The residue was vacuum dried at 45-50° C. Yield: 4-5 g.

Enantiomeric excess=99.74%

m.p.=133-135° C.

[α]_{D} at 25° C.=98.32° (c=5 water)

¹H NMR (DMSO d₆): 2.99-3.07 (dd, 1H), 3.35-3.38 (dd, 1H), 3.72-3.74 (m, 2H), 5.14-5.18 (t, 1H), 5.32-5.38 (t, 1H), 5.71-5.75 (d, 1H), 6.16-6.21 (t, 1H), 7.22-7.27 (d, 2H), 7.80-7.83 (d, 1H)

Moisture content: 1.67%

IR (in KBr, cm⁻¹): 3551, 3236, 2927, 1614, 1492, 1404, 1336, 1253, 1146, 1052, 967, 786.

MS: M+1=230

XRD [2θ] (Cu-K_{α1}=1.54060 Å, K_{α2}=1.54443 Å K_{β}=1.39225 Å; 40 mA, 45 kV): 5.08, 9.89, 10.16, 11.40, 11.65, 12.96, 13.23, 15.26, 15.82, 17.74, 18.74, 18.88, 19.67, 20.69, 22.13, 22.88, 23.71, 25.47, 26.07.

### Example 10

### Preparation of Benzoyloxy Acetaldehyde

### 10a] Preparation of 1,2-isopropylidene glycerol

100 g of glycerol in 400 mL of DCM was treated with 5 g of PTSA and 400 mL of acetone. The reaction mixture was refluxed with concomitant azeotropic removal of water, cooled to room temperature and the solvent was distilled off completely under vacuum (300-400 mm of Hg). The product was distilled at 50-55° C. under 1-2 mm of pressure. Yield: 100-120 g (69-83.6%)

¹H NMR (CDCl₃): δ 1.33 (s, 3H), 1.4 (s, 3H), 2.54 (bs, 1H), 3.50-3.71 (m, 2H), 3.73-3.77 (m, 1H), 3.96-4.03 (t, 1H), 4.03-4.25 (m, 1H)

MS: M+1=133

### 10b] Preparation of benzoylated 1,2-isopropylidene glycerol

135 g (1.02 moles) of 1,2-isopropylidene glycerol in 1.35 L of DCM and 155 g (1.53 moles) of triethylamine was cooled to 5-10° C. and treated with 150.5 g (1.07 moles) of benzoyl chloride dissolved in 270 mL of DCM. When the reaction was complete, 700 mL of DM water was added to the reaction mixture, the organic layer was separated and washed with another 700 mL of DM water. DCM was distilled off completely under a vacuum of 300-400 mm of Hg. 270 mL of THF was added to the residue and then distilled off at 45-50° C., 100-150 mm of Hg to remove the traces of triethylamine. Yield: 230 g (95%)

¹H NMR (CDCl₃): δ 1.35 (s, 3H), 1.42 (s, 3H), 3.80-3.87 (m, 1H), 4.07-4.14 (m, 1H), 4.29-4.49 (m, 3H), 7.25-7.55 (m, 3H), 8.0-8.04 (m, 2H)

MS: m/z=179, 150

### 10c] Preparation of benzoyl glycerol Compound IV

225 g (0.95 moles) of benzoylated 1,2-isopropylidene glycerol dissolved in 1.125 L of THF was heated to 45-50° C. with 45 mL of 2N HCl. When the reaction was complete, the mixture was cooled to room temperature, neutralized with 11 g of NaHCO₃, stirred, and filtered. The THF in the filtrate was distilled under vacuum (100-150m of Hg). Yield: 187 g

Twice stirring it in 1.1 L of 5% ethylacetate/hexane for 30 minutes each time re-crystallized the product.

Yield: 165 g (88%)

MS: M+1=197

¹H NMR (CDCl₃): δ 3.21 (bs, 2H), 3.6-3.74 (m, 2H), 4.03-4.10 (m, 1H), 4.37-4.39 (d, 2H), 7.25-8.03 (m, 5H)

### 10d] Oxidation of Benzoyl glycerol to Benzoyloxy acetaldehyde Compound V

To 160 g (0.816 moles) of Benzoyl glycerol dissolved in 1.6 L of DCM and 80 mL of DM water, 192 g (0.897 moles) of NaIO₄ was added in portions at room temperature. When the reaction was complete, the solid was filtered and the filtrate was concentrated to get the desired product.

MS: M⁺+1=165

¹H NMR (CDCl₃): δ 4.88 (s, 2H), 7.25-8.11 (m, 5H), 9.7 (s, 1H)

### Example 11

### Preparation of 2-Benzoyloxymethyl-5-acetoxy-1,3-oxathiolane

### Compound VIII

100 g (0.609 moles) of benzoyloxy acetaldehyde, 55.7 g (0.365 moles) of 1,4-dithian-2,5-diol and 197.5 g of pyridine are heated at 60-65° C. for 2 hours in a 2.0 L, 4-necked round bottom flask. The reaction mixture is cooled to 0-5° C. and 500 mL of DCM was added to it. 143.6 g (1.83 moles) of acetyl chloride dissolved in 200 mL of DCM was added dropwise to the reaction mixture over a period of 1-2 hours. After the addition was complete, the mass was stirred for 1 hour. After completion of the reaction, the reaction mixture was poured slowly into 500 mL of saturated NaHCO₃ solution. The organic layer was separated and washed with 500 mL of brine. The organic layer was isolated and the solvent was distilled off completely under vacuum (200-250 mm of Hg). 500 mL of cyclohexane and 5 g of activated carbon was added to the residue, stirred for 10 minutes and filtered through a celite bed. The cyclohexane was distilled off completely under vacuum (100-125 mm of Hg, 40-45° C.). Yield: 145-155 g (84.3-90% w/w)

¹H NMR (CDCl₃): δ 2.0 (s, 3H), 3.04-3.30 (m, 2H), 4.36-4.49 (m, 2H), 5.57-5.62 (m, 1H), 6.54-6.64 (dd, 1H), 7.31-7.48 (m, 3H), 7.94-7.98 (m, 2H)

### Example 12

### Preparation of cis- and trans-2-Benzoyloxymethyl-5-cytosin-1′-yl-1,3-oxathiolane

### Compound IX

76.8 g (0.69 moles) of cytosine, 652.4 g (842 mL, 4.04 moles) of HMDS and 7.5 g (0.07 moles) of TMSCl were taken in a 3.0 L, 4-necked round bottom flask under nitrogen atmosphere. The contents were refluxed at 125-130° C. for 1-2 hours until a clear solution was obtained. The mass was cooled to 100° C. and the reagents in excess were distilled off completely under vacuum. 150 g (0.53 moles) of benzoyloxymethyl-acetoxy-1,3-oxathiolane, 1.5 L of DCM and 165.5 g (0.74 moles) of TMSTf were added to the residue. (Instead of TMSTf, 148.5 g of TMS-I may be used.) The reaction mixture was refluxed and when the reaction was complete, it was cooled to 0° C. and charged with 750 mL of DM water. (The initial 100 mL were added drop wise.) After stirring for 15 minutes, the organic layer was separated, washed with 1.5 L of saturated NaHCO₃ solution and DCM was distilled off completely under vacuum (200 mm of Hg, 30-35° C.). Yield: 150-160 g (84.7-90.3%)

MS: M⁺+1=334

¹H NMR (DMSO d₆): δ 3.10 (dd, 1H), 3.48 (dd, 1H), 4.64 (d, 2H), 5.50 (t, 1H), 5.6 (d, 1H) 6.25 (t, 1H), 7.27 (d, 2H), 7.50-7.72 (m, 4H), 7.95-7.98 (m, 2H)

### Example 13

### Separation of Cis/Trans-2-Benzoyloxymethyl-5-cytosin-1′-yl-1,3-oxathiolane

### a] Using S-(+)-Mandelic Acid

5 g (0.015 moles) of cis- and trans-2-Benzoyloxymethyl-5-cytosin-1′-yl-1,3-oxathiolane and 100 mL MeOH were heated till a clear solution was obtained. 2.3 g (0.015 moles) of (+)-Mandelic acid was added and the contents were heated to 65-66° C. for the compound to dissolve. The reaction mixture was stirred overnight at room temperature and the solid was filtered, washed with 5 mL of cold MeOH to yield the Cis isomer. Yield: 2 g (50.52% yield cis racemate)

### b] Using Succinic Acid

5 g (0.015 moles) of cis- and trans-2-Benzoyloxymethyl-5-cytosin-1′-yl-1,3-oxathiolane and 140 mL MeOH were heated till a clear solution was obtained. 1.77 g (0.015 moles) of succinic acid was added and the contents were heated to 65-66° C. for the compound to dissolve. The reaction mixture was stirred overnight at room temperature and the solid was filtered, washed with 5 mL of cold MeOH to yield the Cis isomer. Yield: 1.5 g (40.8% yield cis racemate)

### c] Using Oxalic Acid

5 g (0.015 moles) of cis- and trans-2-Benzoyloxymethyl-5-cytosin-1′-yl-1,3-oxathiolane and 50 mL MeOH were heated till a clear solution was obtained. 2.83 g (0.022 moles) of oxalic acid was added and the contents were heated to 65-66° C. for the compound to dissolve. The reaction mixture was stirred overnight at room temperature and the solid was filtered, washed with 5 mL of cold MeOH to yield the Cis isomer. Yield: 1.0 g (28.86% yield cis racemate)

### d] Using Dichloromethane

5 g of cis- and trans-2-Benzoyloxymethyl-5-cytosin-1′-yl-1,3-oxathiolane was dissolved in 25 mL of DCM and heated to 65-66° C. The solution was stirred overnight at room temperature and the solid was filtered to yield the Cis isomer.

Yield: 1.0 g (36.44% yield cis racemate)

After separation of isomers, the free base is prepared by a procedure as detailed in Example 6.

### Example 14

### Debenzoylation (Lamivudine Cis±)

### Compound XII

16 g of Cis-(±)-2-Benzoyloxymethyl-5-cytosin-1′-yl-1,3-oxathiolane was treated with 320 mL of methanolic ammonia at room temperature. The reaction mixture was stirred for 2 hours and after the reaction was complete the solvent was distilled off completely under vacuum. The residue was charged with 40 mL of ethanol, stirred for 1 hour at room temperature, filtered and dried under vacuum at 40-45° C. Yield: 9.0 g (81.81%)

Purity by HPLC=99.13%

MS: M+1=230

¹H NMR (DMSO d₆): δ 2.98-3.07 (dd, 1H), 3.34-3.43 (dd, 1H), 3.70-3.83 (m, 2H), 5.13-5.17 (t, 1H), 5.36 (bs, 1H), 5.75-5.79 (d, 1H), 6.13-6.18 (t, 1H), 7.21 (d, 2H), 7.83-7.86 (d, 1H).

1.-17. (canceled)

18. A process for the preparation of essentially enantiomerically pure (−)-[2R,5S]-4-amino-1-[2-(hydroxymethyl)-1,3-oxathiolan-5-yl]-2(1H)-pyrimidin-2-one of formula (I), 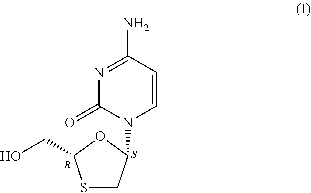
from a compound of formula IX, 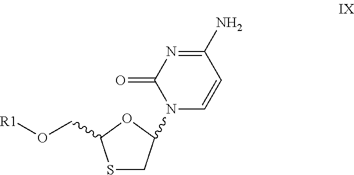
the process comprising the steps of:
I) preparation of (±)-1-(2R/S-Cis)-4-amino-1-[(2-hydroxymethyl)-1,3-oxathiolan-5-yl]-2(1H)-pyrimidin-2-one of formula (XII), 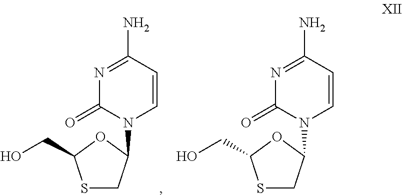
from a mixture of cis-(±) and trans-(±) intermediate of Formula (IX) 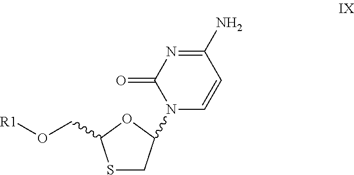
wherein, R1 is alcohol protecting group
comprising the steps of:
a) treating compound of formula IX with an acid in an organic solvent selected from C₁ to C₈ alcohol, C₃ to C₁₀ ester, C₁ to C₄ haloalkane and mixtures thereof to form a salt;
b) isolating the acid addition salt of the cis-(±) isomers of formula (X), by filtration; 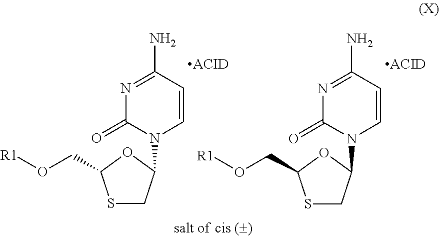
c) converting the salt obtained in step b) to its free base by treatment of base like aqueous ammonia;
d) deprotecting the free base obtained in step c) to the compound of Formula XII;
II) preparation of essentially enantiomerically pure (−)-[2R,5S]-4-amino-1-[2-(hydroxymethyl)-1,3-oxathiolan-5-yl]-2(1H)-pyrimidin-2-one of formula (I), 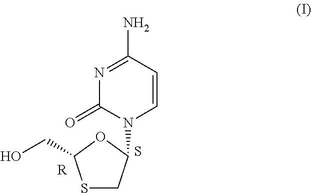
from a mixture of racemic cis-(±)-Lamivudine of formula (XII), 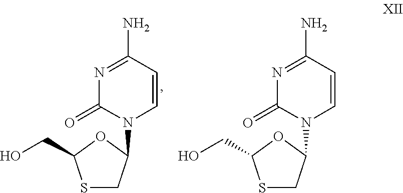
comprising the steps of:
a) treating (±)-1-(2R/S-Cis)-4-amino-1-[(2-hydroxymethyl)-1,3-oxathiolan-5-yl]-2(1H)-pyrimidin-2-one of formula (XII) with S-Binol in an organic solvent;
b) isolating the adduct formed by the cis (−) enantiomer and S-Binol;
c) treating the adduct with hydrochloric acid; and
d) neutralizing the reaction mixture formed after c) using sodium hydroxide, thereby obtaining enantiomerically pure (−)-[2R,5S]-4-amino-1-[2-(hydroxymethyl)-1,3-oxathiolan-5-yl]-2(1H)-pyrimidin-2-one of formula (I).

19. The process according to claim 18, wherein the organic solvent comprises C1 to C8 alcohol.

20. The process according to claim 18, wherein R1 is trialkylsilyl or C₂ to C₉ acyl.

21. The process according to claim 19, wherein R1 is tertiary-butyldiphenylsilyl or benzoyl.

22. The process according to claim 18, wherein the acid is selected from Succinic acid, Oxalic acid, [S]-(+)-Mandelic acid, Di-para-toluoyl-D-Tartaric acid and 1S-(+)-10-camphorsulfonic acid.

23. The process according to claim 18, wherein the ratio of the compound of formula (IX) to the acid is 1:1 to 1:2.

24. The process according to claim 18, wherein the reaction (I) is carried out at a temperature between 20 and 40° C.

25. The process according to claim 24 wherein the reaction (I) is carried out between 25 and 30° C.

26. A process according to claim 18, wherein the compound of formula (X) is purified using methanol before proceeding to step I) c).

27. A process for the preparation of essentially enantiomerically pure (−)-[2R,5S]-4-amino-1-[2-(hydroxymethyl)-1,3-oxathiolan-5-yl]-2(1H)-pyrimidin-2-one of formula (I), 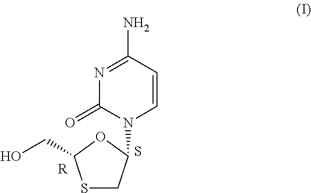
from a compound of formula IX, 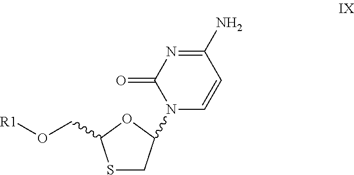
the process comprising the steps of:
I) preparation of (±)-1-(2R/S-Cis)-4-amino-1-[(2-hydroxymethyl)-1,3-oxathiolan-5-yl]-2(1H)-pyrimidin-2-one of formula (XII), 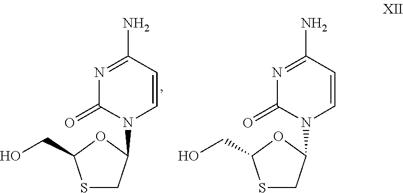
from a mixture of cis-(±) and trans-(±) intermediate of Formula (IX) 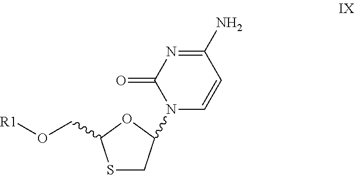
comprising the steps of:
a) dissolving compound of formula IX in an organic solvent such as dichloromethane at about 65° C.;
b) stirring a solution formed in a) at an ambient temperature for twelve hours;
c) isolation of the product solidified;
d) deprotecting the product obtained in step c) to the compound of Formula XII;
II) Preparation of essentially enantiomerically pure (−)-[2R,5S]-4-amino-1-[2-(hydroxymethyl)-1,3-oxathiolan-5-yl]-2(1H)-pyrimidin-2-one of formula (I), 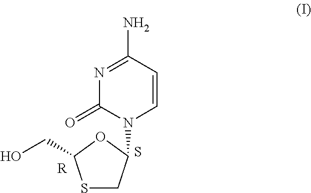
from a mixture of racemic cis-(±)-Lamivudine of formula (XII), 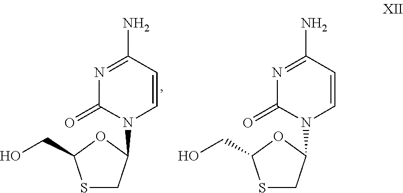
comprising the steps of:
a) treating (±)-1-(2R/S-Cis)-4-amino-1-[(2-hydroxymethyl)-1,3-oxathiolan-5-yl]-2(1H)-pyrimidin-2-one of formula (XII) with S-Binol in an organic solvent;
b) isolating the adduct formed by the enantiomer and S-Binol;
c) treating the adduct with hydrochloric acid;
d) neutralizing the reaction mixture formed after ‘(c)’ using sodium hydroxide, thereby obtaining enantiomerically pure (−)-[2R,5S]-4-amino-1-[2-(hydroxymethyl)-1,3-oxathiolan-5-yl]-2(1H)-pyrimidin-2-one of formula (I).

28. The process according to claim 27, wherein the organic solvent comprises C1 to C8 alcohol.

29. A process for the preparation of essentially enantiomerically pure (−)-[2R,5S]-4-amino-1-[2-(hydroxymethyl)-1,3-oxathiolan-5-yl]-2(1H)-pyrimidin-2-one of formula (I), 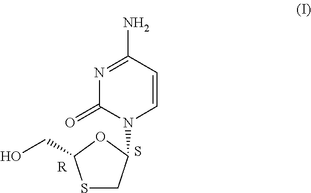
from (±)-1-(2R/S-Cis)-4-amino-1-[(2-hydroxymethyl)-1,3-oxathiolan-5-yl]-2(1H)-pyrimidin-2-one of formula (XII), 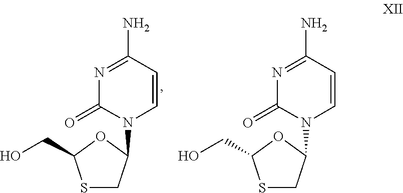
comprising the steps of:
a) treating (±)-1-(2R/S-Cis)-4-amino-1-[(2-hydroxymethyl)-1,3-oxathiolan-5-yl]-2(1H)-pyrimidin-2-one of Formula (XII) with S-Binol in an organic solvent;
b) isolating the adduct formed by the enantiomer and S-Binol;
c) treating the adduct with an acid;
d) neutralizing the reaction mixture formed after c) using a base, thereby obtaining enantiomerically pure (−)-[2R,5S]-4-amino-1-[2-(hydroxymethyl)-1,3-oxathiolan-5-yl]-2(1H)-pyrimidin-2-one of formula (I).

30. The process according to claim 29, wherein the organic solvent is a C₁ to C₈ alcohol.

31. The process according to claim 30, wherein the organic solvent is methanol.

32. The process according to claim 29, wherein the ratio of the compound of formula (XII) to S-Binol is 1:1 to 1:2.

33. The process according claim 29, wherein in step c), the adduct is treated with hydrochloric acid.

34. The process according to claim 29, wherein the base used to neutralize the salt is Sodium hydroxide.
